(19) United States
(12) Patent Application Publication
(10) Pub. No.: US 2026/0215909 A1
(43) Pub. Date: Jul. 30, 2026

(54) LENSES CHANGING OPTICAL PERFORMANCE IN RESPONSE TO AN ACCOMMODATIVE STIMULUS

(71) Applicant: AMO Groningen B.V., Groningen (NL)

(72) Inventors: Hendrik Weeber, Groningen (NL); Marrie van der Mooren, Groningen (NL); Sieger Meijer, Groningen (NL); Carmen Canovas Vidal, Groningen (NL); Antonio DEL ÁGUILA CARRASCO, Groningen (ES); Aixa ALARCON HEREDIA, Groningen (ES)

(21) Appl. No.: 19/148,996

(22) PCT Filed: Jan. 17, 2024
(86) PCT No.: PCT/IB2024/050441

§ 371 Date: Jul. 17, 2025

Related U.S. Application Data
(60) Provisional application No. 63/480,211, filed on Jan. 17, 2023.

(51) Int. Cl. A61F 2/16

(52) U.S. Cl. CPC A61F 2/1627; A61F 2/1654; A61F 2002/1682; A61F 2250/0003; A61F 2250/0013

(57) ABSTRACT

Improved ophthalmic lenses, particularly intraocular lenses (IOLs). Examples may include lenses configured to have optical performance that varies in response to an accommodative stimulus.

### CROSS REFERENCE TO RELATED APPLICATIONS

This application claims the benefit of U.S. Provisional Application No. 63/480,211, filed Jan. 17, 2023, the entire contents of which is hereby incorporated by reference.

### FIELD

The present disclosure relates to lenses, particularly intraocular lenses (IOLs).

### BACKGROUND

A human eye can suffer diseases that impair a patient's vision. For instance, a cataract may increase the opacity of the lens, causing blindness. To restore the patient's vision, the diseased or damaged lens may be surgically removed and replaced with an artificial lens, known as an intraocular lens, or IOL. An IOL may also be used for presbyopic lens exchange or other elective ocular surgical procedures.

Monofocal IOLs are intended to provide vision correction at one distance only, usually the far focus. At the very least, since a monofocal IOL provides vision treatment at only one distance and since the typical correction is for far distance, spectacles are usually needed for good vision at near distances and sometimes for good vision at intermediate distances. The term “near vision” generally corresponds to vision provided when objects are at a distance from the subject eye at equal; or less than 1.5 feet. The term “distance vision” generally corresponds to vision provided when objects are at a distance of at least about 5-6 feet or greater. The term “intermediate vision” corresponds to vision provided when objects are at a distance of about 1.5 feet to about 5-6 feet from the subject eye. Such characterizations of near, intermediate, and distance vision correspond to those addressed in Morlock R, Wirth R J, Tally S R, Garufis C, Heichel C W D, Patient-Reported Spectacle Independence Questionnaire (PRSIQ): Development and Validation. Am J Ophthalmology 2017; 178:101-114.

There have been various attempts to address limitations associated with monofocal IOLs. For example, multifocal IOLs have been proposed that deliver, in principle, two foci, one near and one far, optionally with some degree of intermediate focus. Such multifocal, or bifocal, IOLs are intended to provide good vision at two distances, and include both refractive and diffractive multifocal IOLs. In some instances, a multifocal IOL intended to correct vision at two distances may provide a near (add) power of about 2.5 or 4.0 diopters.

Multifocal IOLs may, for example, rely on a diffractive optical surface to direct portions of the light energy toward differing focal distances, thereby allowing the patient to clearly see both near and far objects. Depending on the configuration, multifocal IOLs may also allow a patient to see intermediate vision. More recently diffractive optical surfaces have created extended depth of focus and/or extended range of vision lenses which allow for continuous vision from far to near. Diffractive optical surfaces may also be configured to provide reduced chromatic aberration. Multifocal lenses (including contact lenses or the like) have also been proposed for treatment of presbyopia without removal of the natural crystalline lens.

Current state of the art diffractive monofocal, extended depth of focus (EDOF), and multifocal lenses, and in general, non-accommodative lenses providing a full range of vision, can make use of a material having a given refractive index and a surface curvature which provide a refractive power. Diffractive lenses have a diffractive profile which confers the lens with diffractive powers that may contribute to the overall optical power of the lens. The diffractive profile is typically characterized by a number of diffractive zones. When used for ophthalmic lenses these zones are typically annular lens zones, or echelettes, spaced about the optical axis of the lens. One or more, or each echelette may be defined by an optical zone, a transition zone, and an echelette geometry. The echelette geometry includes an inner and outer diameter and a shape or slope of the optical zone, a height or step height, and a shape of the transition zone. The surface area or diameter of the echelettes largely determines the diffractive power(s) of the lens. The slope of the optical zone is the gradient of the diffractive profile, e.g. the gradient varies across the echelette. The height, the width and the shape of the transition zone between echelettes largely determines the light distribution between the different powers or diffractive orders. Together, these echelettes form a diffractive profile.

Although multifocal ophthalmic lenses lead to improved quality of vision for many patients, additional improvements may be beneficial. IOLs may comprise accommodating IOLs, which may produce varied characteristics in response to an accommodative stimulus from an eye. Accommodative IOLs are characterized by a mechanism that changes the refractive optical power of the eye in response to an accommodative stimulus. In many embodiments of accommodative IOLs the accommodative stimulus is the contraction of the ciliary muscles in the eye. For example, an accommodating IOL may adjust its axial position, shape, and/or thickness to effect an optical power change of the eye within a particular range, similar to the eye's natural lens.

This ability to accommodate may be of benefit for a patient, and may more closely approximate a patient's natural vision than a nonaccommodative IOL. In addition, it would be beneficial if the accommodative IOL is comprised of a surface that may turn into a diffractive lens in response to an accommodative stimulus. When there is not an accommodative stimulus, the lens may be a refractive lens.

### BRIEF SUMMARY

Examples herein described may be directed to lenses, particularly intraocular lenses (IOLs).

Examples herein described may be directed to an intraocular lens for implantation within an eye. The intraocular lens may be configured to produce varied characteristics in response to an accommodative contraction of the eye. The intraocular lens may be configured to comprise a monofocal lens when the eye is configured for distance vision and configured to increase a depth of focus or range of vision in response to the accommodative contraction of the eye without significant changing of the dioptric power of the eye. Examples herein described include an apparatus comprising: an intraocular lens for implantation within an eye, the intraocular lens configured to produce varied optical characteristics in response to a stimulus, the intraocular lens configured to comprise a monofocal lens when the eye is configured for distance vision and configured to change a depth of focus or range of vision in response to a stimulus with less than a 1 diopter change of dioptric power of the eye, wherein the depth of focus or range of vision is caused in part by diffraction.

Examples herein described include an apparatus comprising an intraocular lens for implantation within an eye, the intraocular lens configured to produce varied optical characteristics in response to a stimulus, the intraocular lens configured to comprise a monofocal lens when the eye is configured for distance vision and configured to change a depth of focus or range of vision in response to a stimulus with less than a 1.3 diopter change of dioptric base power of the eye, wherein the depth of focus or range of vision is caused in part by diffraction.

Examples herein described include an apparatus comprising: an intraocular lens for implantation within an eye, the intraocular lens configured to produce varied optical characteristics in response to a stimulus, the variation generated by a deformation of a material having a sinusoidal stiffness gradient.

The sinusoidal stiffness gradient may be located in an anterior wall and/or a posterior wall of the intraocular lens. The gradient stiffness may be sinusoidal in a radial direction. The period of the sinusoidal stiffness gradient may be reduced with a square of a radial coordinate. It is further envisioned that the sinusoidal stiffness gradient may extend for only a portion of the optic.

The intraocular lens may have a central portion that may be filled with a fluid or gel. It is also envisioned that the intraocular lens may have one or more haptics configured to be filled with a fluid or gel which may transfer the fluid or gel to or from a chamber in the central portion.

### BRIEF DESCRIPTION OF THE DRAWINGS

FIG. 1 is a plan drawing of a human eye having an implanted intraocular lens in an accommodative or “near” state.

FIG. 2 is a plan drawing of the human eye of FIG. 1 in a disaccommodative or “far” state.

FIG. 3 is a side cross sectional view of an intraocular lens.

FIG. 4 is a graph of the sinusoidal stiffness gradient in the wall of the anterior optic in FIG. 3.

FIG. 5 is a graph of a shape of an inner side of an anterior wall of an optic according to examples herein.

FIG. 6 is a graph of a shape of an inner side of an anterior wall of an optic according to examples herein.

### DETAILED DESCRIPTION

In a healthy human eye, the natural lens is housed in a structure known as the capsular bag. During natural accommodation, the capsular bag is driven by a ciliary muscle and zonular fibers (also known as zonules) in the eye, which can pull on the capsular bag to change its shape. The change in shape of the capsular bag generally deforms the natural lens in order to change its power and/or the location of the lens, so that the eye can focus on objects at varying distances away from the eye in a process known as accommodation.

Accommodation is defined as the change in the dioptric power of an eye (Bennett, A. G., & Rabbetts, R. B. (1989). Clinical Visual Optics. (p. 135). Oxford: Butterworth-Heinemann), and comprises a change in refraction of an eye.

Natural accommodation may occur when an eye focus on an object at intermediate or near distances, which may induce contraction of the ciliary muscle, which may cause a change in diameter, thickness, radius of curvature and/or power of the natural lens. Along with these changes, accommodation may also include a change in vergence of the eyes and change (decrease) the pupil diameter. Contraction of the ciliary muscle, changes in vergences and in pupil size may also occur in pseudophakic eyes, irrespective of whether the eye changes its dioptric power.

Examples as disclosed herein may be directed to lenses that produce varied optical characteristics in response to an accommodative effort of an eye, for example, a contraction of the ciliary muscle of an eye. The intraocular lenses may advantageously use ocular forces, such as those produced by the ciliary muscle, zonules, and/or capsular bag, to change the shape of the lens optic or otherwise vary the optical characteristics of the intraocular lens.

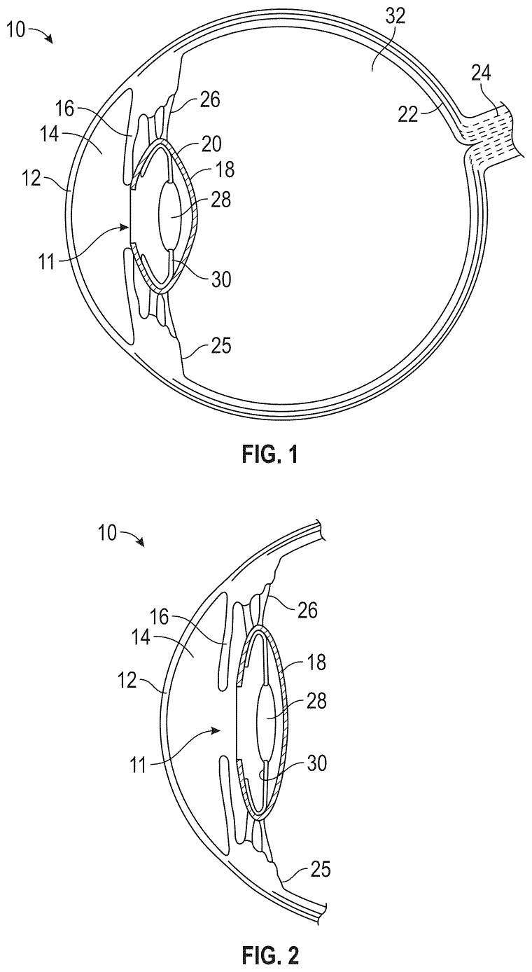

FIG. 1 shows a human eye 10, after an intraocular lens 11 has been implanted. Prior to surgery, the natural lens occupies essentially the entire interior of the capsular bag 18. After surgery, the capsular bag 18 may house the intraocular lens 11. Alternatively, an intraocular lens may be configured to directly engage the zonules or ciliary muscle in examples herein. For example, an intraocular lens may be placed in the sulcus or other position as desired.

Light enters the eye 10 from the left in FIG. 1 and passes through the cornea 12, the anterior chamber 14, the pupil (defined by the inner edge of the iris 16), and impinges on the intraocular lens 11. After passing through the intraocular lens 11, light exits the posterior wall 20 of the capsular bag 18, passes through the vitreous body 32, and strikes the retina 22, which detects the light and converts it to a signal transmitted through the optic nerve 24 to the brain.

A well-corrected eye focuses an image at the retina 22. If the intraocular lens 11 has too much or too little power, the focused image shifts axially along the optical axis off of the retina, toward or away from the lens 11. Note that the total power of the eye (e.g., including the combined power of cornea 12 and the intraocular lens 11) required to focus on a close or near object is higher than the power required to focus on a distant or far object. The difference between the “near power” and “far power” is known typically as the range of accommodation or the add power. A typical range of accommodation or add power is about 2 to 4 diopters, but may be significantly larger for younger human subjects.

The capsular bag 18 is acted upon by the ciliary muscle 25 and the zonules 26, which distort the capsular bag 18 by stretching it radially in a relatively thick band about its equator. Experimentally, it is found that the ciliary muscle 25 and/or the zonules 26 typically exert a total force of up to about 10 grams of force, which is generally distributed uniformly around an equatorial region of the capsular bag 18. In some patients, non-uniform forces may be applied to the capsular bag 18, for example, due to damage of the zonules.

The intraocular lens 11 generally has an optic 28 made of a transparent, deformable and/or elastic material and may include a haptic 30 configured to hold the optic 28 in place and to mechanically transfer forces from the eye (e.g., from the capsular bag 18 or ciliary muscle 25) to the optic 28. A haptic 30 may have an engagement member with a central recess that is sized to receive the peripheral edge of the optic 28. Other forms of haptics may be utilized.

When the eye 10 is focused on a relatively close object, as shown in FIG. 1, the ciliary muscle 25 is compressed, which causes the zonules 26 to relax and allow the equatorial region of the capsular bag 18 to contract. The capsular bag 18 in this state is thicker at its center and has more steeply curved sides.

FIG. 2 shows a portion of the eye 10 focused on a relatively distant object. To focus on the distant object, the zonules 26 are retracted and the shape of the capsular bag 18 is thinner at its center and has less steeply curved sides.

The configuration of the eye 10 shown in FIG. 2 may be a configuration for distance vision. The configuration of the eye 10 shown in FIG. 1 may be a configuration for near vision. An accommodative contraction of the eye 10 may occur to move the lens 11 from the configuration shown in FIG. 2 to the configuration shown in FIG. 1.

Current state of the art non-accommodative intraocular lenses can be multifocal lenses, extended depth of focus lenses, or combinations thereof. These lenses are sometimes called simultaneous vision lenses, as the provide useful vision over a range of vision simultaneously through optical means. While simultaneous vision lenses may provide adequate visual acuity of a range of distances, they have the general drawback of suffering from a reduced contrast and an increase of dysphotopsia. These drawbacks play a role predominantly when the eye is configured for distance vision. According to examples herein, an intraocular lens may be provided that may provide a full range of vision without having side effects of contrast loss and dysphotopsia for distance vision.

According to examples herein, an intraocular lens may be provided for implantation within an eye 10, with the intraocular lens configured to produce varied optical characteristics in response to a stimulus. The stimulus may comprise an ocular accommodative effort of the eye or accommodative stimulus that is accompanied by the contraction of the ciliary muscle of the eye 10. The intraocular lens may be configured to comprise a monofocal lens when the eye is configured for distance vision. The eye, for example, may have relaxed ciliary muscles 25 when the eye is configured for distance vision. An optic of an intraocular lens may be purely monofocal in such a configuration. With an optic being monofocal, a reduced possibility of dysphotopsia (e.g., comparable to a standard monofocal IOL) may result, and there may be a reduced possibility of loss of contrast.

According to examples herein, the intraocular lens may be configured to change a depth of focus or range of vision in response to the stimulus (that is e.g. accompanied by the contraction of the ciliary muscle). The change in response to the accommodative effort may occur without significantly changing the refraction or power of the eye. For example, a less than 1 diopter change of dioptric power of the eye 10 may occur. The intraocular lens may be configured to not significantly change the refraction of the eye in the near vision configuration of the eye. The intraocular lens may be configured to change the optical characteristics in response to an accommodative stimulus (e.g., contraction of the ciliary muscle 25), without significantly changing the base power of the lens (e.g., less than a 1.3 diopter change in the base power of the lens). The changed optical characteristics may allow for improved intermediate and near vision as compared with a standard monofocal IOL having the same base power.

According to examples herein, the intraocular lens may be configured to comprise an extended depth of focus or range of vision lens when the eye 10 is configured for near vision. The eye 10 may have contracted ciliary muscles 25 when the eye is configured for near vision.

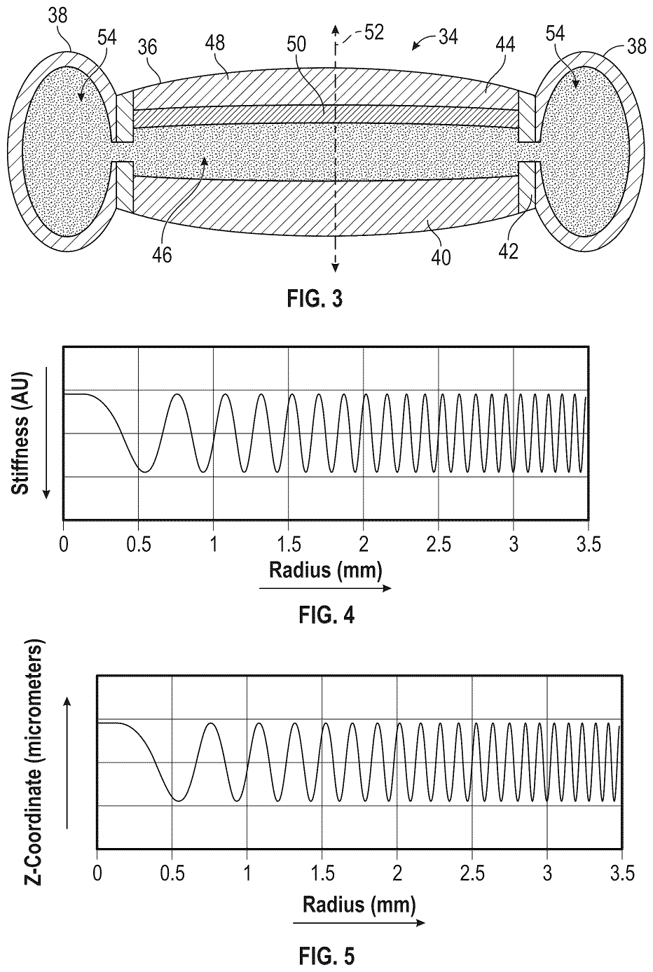

FIG. 3 illustrates a cross sectional view of an intraocular lens 34 including an optic 36. The intraocular lens 34 may include one or more haptics 38 that may be positioned at a periphery of the optic 36 or at another position as desired.

The optic 36 may include one or more walls 40, 42, 44. The one or more walls 40, 42, 44 may bound a central portion or chamber 46 of the optic 36. The chamber 46 may be configured to be filled with a fluid or a gel. It is also envisioned that the chamber 46 may be configured to be filled with incompressible materials as desired. The one or more walls may include a posterior wall 40, a side wall or peripheral wall 42, and an anterior wall 44. The central portion or chamber 46 is positioned between the posterior wall 40 and the anterior wall 44, and is bound on its sides by the side wall or peripheral wall 42.

The anterior part 48 of the anterior wall 44 may be relatively stiffer than the posterior part 50 of the anterior wall. The posterior part 50 of the anterior wall 44 may contain a sinusoidal stiffness gradient with varying stiffness. The varying stiffness of the sinusoidal stiffness gradient may produce diffraction during accommodation. The diffraction may, at least in part, cause multifocality, a depth of focus, or an extended range of vision.

It is also envisioned that the posterior part of the posterior wall 40 may be relatively stiffer than the anterior part of the posterior wall. It is also envisioned that the posterior optic wall 40 may contain a sinusoidal stiffness gradient on an anterior side of the posterior wall 40. The anterior wall 44 and/or posterior wall 40 may include the stiffness gradient (e.g., the sinusoidal stiffness gradient).

The central portion or chamber 46 of the lens is fluid or gel filled where the fluid or gel flows into the optic 36 following a stimulus (e.g., an accommodative stimulus). There may be a difference in refractive index between the optic wall and the fluid or gel of at least 0.01. The difference can be either positive or negative.

The wall of the fluid/gel filled optic may include the gradient stiffness. The gradient stiffness is characterized by being rotationally symmetric around the optical axis. The gradient stiffness is further characterized by being sinusoidal in the radial direction, and the period of the sinusoidal stiffness reduces with the square of the radial coordinate.

FIG. 4 shows the (gradient) stiffness as a function of the radial coordinate of the optic 36, with the point of zero radius indicated by the optical axis 52 in FIG. 3. FIG. 5, for example, shows the shape of the inner side or posterior part 50 of the anterior wall 44 of the optic 36. The shape is that of a sinusoidal diffractive multifocal lens.

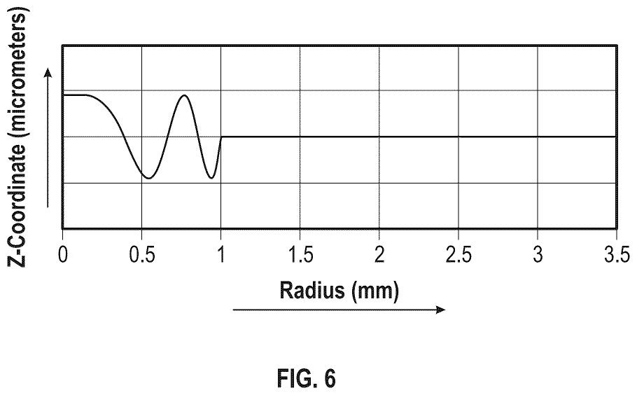

The diffractive structure or profile may cover the entirety of the optic (e.g., from the optical axis 52 to the end of the 3.5 millimeter radius represented in FIG. 5). In examples, the diffractive structure or profile may also cover only a portion of the optic 36. The portion of the optic 36 can be any concentric portion of the optic 36, or include multiple concentric portions of the optic 36. FIG. 6, for example, shows the diffractive structure or diffractive profile being placed in a central portion of the optic 36 at the optical axis 52. In specific embodiments the diffractive structure or profile may include 1 diffractive zone or ring. In alternative embodiments the structure or profile may include at least 2 rings at least 3 rings, or at least 4 rings.

It is further envisioned that for the diffractive structure or profile, rather than a sinusoidal shape, a sine-cosine function, a step function, and any other diffractive structure as known in the art may be implemented.

The one or more haptics 38 may include one or more chambers 54. The chambers 54 may be configured to be filled with fluid or gel and may be configured to transfer fluid or gel to or from the chamber 46 of the optic 36. The one or more haptics 38 may be made of a flexible material. For example, the one or more haptics 38 may be configured to be compressed towards the optic 36 upon a contraction of the eye occurring. The one or more haptics 38 may be retracted radially away from the optic 36 and the chambers 54 may be expanded upon an accommodative relaxation of the eye. As such, upon accommodative relaxation of the eye, the haptics 38 may be configured to transfer fluid or gel from the chamber 46 into the chambers 54. Upon an accommodative stimulus and contraction of the ciliary muscle of the eye, the haptics 38 may be configured to transfer fluid or gel from the chambers 54 of the haptics 38 to the chamber 46 of the optic 36. The material comprising the gradient stiffness (e.g., the diffractive part or layer) of the optic 36 may be deformed in response to the stimulus (e.g., the accommodative stimulus). For example, the material comprising the gradient stiffness may bow or deflect outward from the chamber 46 upon fluid or gel entering the chamber 46, and may bow or deflect inward towards the chamber 46 upon fluid or gel exiting the chamber 46 into the chambers 54. The varied optical characteristics disclosed herein are produced by the deformation of the material having the gradient stiffness. The diffractive profile on the material having the gradient stiffness deforms to produce the varied optical characteristics disclosed herein.

The lenses disclosed herein may be configured to provide a full range of vision with a reduced possibility of adverse side effects (e.g., loss in contrast and dysphotopsia). These adverse effects may be reduced for distance vision, at which such effects typically occur. The lenses disclosed herein may be configured to address such adverse side effects.

While the present disclosure has been described with respect to various specific examples and embodiments, it is to be understood that the disclosure is not limited thereto and that it can be variously practiced within the scope of the following claims. Combinations of features across various examples may result.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. An apparatus comprising:
an intraocular lens for implantation within an eye, the intraocular lens configured to produce varied optical characteristics in response to a stimulus, the intraocular lens configured to comprise a monofocal lens when the eye is configured for distance vision and configured to change a depth of focus or range of vision in response to a stimulus with less than a 1 diopter change of dioptric power of the eye, wherein the depth of focus or range of vision is caused in part by diffraction.

2. The apparatus of claim 1, wherein the stimulus is an ocular accommodative effort of the eye.

3. The apparatus of claim 1 or claim 2, wherein the diffraction results from a sinusoidal stiffness gradient.

4. The apparatus of claim 3, wherein the intraocular lens includes an anterior wall and a posterior wall, and the sinusoidal stiffness gradient is located in the anterior wall and/or the posterior wall.

5. The apparatus of claim 4, wherein a central portion of the intraocular lens is positioned between the anterior wall and the posterior wall.

6. The apparatus of claim 5, wherein the central portion is configured to be filled with fluid or gel.

7. The apparatus of claim 6, wherein the central portion comprises a chamber configured to be filled with the fluid or the gel.

8. The apparatus of claim 7, wherein the intraocular lens includes one or more haptics configured to be filled with fluid or gel and configured to transfer fluid or gel to or from the chamber.

9. The apparatus of any of claims 1-8, wherein the intraocular lens includes an anterior wall and a posterior wall and a chamber positioned between the anterior wall and the posterior wall, and a posterior part of the anterior wall includes a material having a gradient stiffness.

10. The apparatus of claim 9, wherein an anterior part of the anterior wall has a greater stiffness than the material having the gradient stiffness.

11. The apparatus of claim 9 or claim 10, wherein the posterior wall has a greater stiffness than the material having the gradient stiffness.

12. The apparatus of any of claims 9-11, wherein the gradient stiffness is sinusoidal in a radial direction.

13. The apparatus of claim 12, wherein a period of the sinusoidal stiffness gradient reduces with a square of a radial coordinate.

14. The apparatus of claim 12 or claim 13, wherein the anterior wall comprises an optic, and the sinusoidal stiffness gradient extends for only a portion of the optic.

15. The apparatus of any of claims 1-14, wherein the intraocular lens includes an anterior wall and a posterior wall and a chamber positioned between the anterior wall and the posterior wall, and a posterior part of the anterior wall includes a diffractive profile.

16. An apparatus comprising:
an intraocular lens for implantation within an eye, the intraocular lens configured to produce varied optical characteristics in response to a stimulus, the intraocular lens configured to comprise a monofocal lens when the eye is configured for distance vision and configured to change a depth of focus or range of vision in response to a stimulus with less than a 1.3 diopter change of dioptric base power of the eye, wherein the depth of focus or range of vision is caused in part by diffraction.

17. The apparatus of claim 16, wherein the stimulus is an ocular accommodative effort of the eye.

18. The apparatus of claim 16 or claim 17, wherein the diffraction results from a sinusoidal stiffness gradient.

19. The apparatus of claim 18, wherein the intraocular lens includes an anterior wall and a posterior wall, and the sinusoidal stiffness gradient is located in the anterior wall and/or the posterior wall.

20. The apparatus of claim 19, wherein a central portion of the intraocular lens is positioned between the anterior wall and the posterior wall, and the central portion is configured to be filled with fluid or gel.

21. An apparatus comprising:
an intraocular lens for implantation within an eye, the intraocular lens configured to produce varied optical characteristics in response to a stimulus, the variation generated by a deformation of a material having gradient stiffness.

22. The apparatus of claim 21, wherein the stimulus is an ocular accommodative effort of the eye.

23. The apparatus of claim 21 or claim 22, wherein the intraocular lens includes an anterior wall and a posterior wall, and the material having the gradient stiffness is located in the anterior wall and/or the posterior wall.

24. The apparatus of claim 23, wherein the intraocular lens includes a chamber positioned between the anterior wall and the posterior wall, and a posterior part of the anterior wall includes the material having the gradient stiffness.

25. The apparatus of any of claims 21-24, wherein the gradient stiffness is sinusoidal in a radial direction.

26. An apparatus comprising:
an intraocular lens for implantation within an eye, the intraocular lens configured to produce varied optical characteristics in response to a stimulus, the variation generated by a deformation of a diffractive profile on a material having gradient stiffness.

27. The apparatus of claim 26, wherein the stimulus is an ocular accommodative effort of the eye.

28. The apparatus of claim 26 or claim 27, wherein the intraocular lens includes an anterior wall and a posterior wall, and the material having the gradient stiffness is located in the anterior wall and/or the posterior wall.

29. The apparatus of claim 28, wherein the intraocular lens includes a chamber positioned between the anterior wall and the posterior wall, and a posterior part of the anterior wall includes the material having the gradient stiffness.

30. The apparatus of any of claims 26-29, wherein the diffractive profile includes at least one diffractive ring.
